(19) United States
(12) Patent Application Publication
(10) Pub. No.: US 2002/0123132 A1
(43) Pub. Date: Sep. 5, 2002

(54) Biochip reader

(76) Inventors: Takeo Tanaami, Tokyo (JP); Akira Ichihara, Tokyo (JP)

Correspondence Address: MOONRAY KOJIMA, BOX 627, WILLIAMSTOWN, MA, 01267, US

(21) Appl. No.: 09/798,605

(22) Filed: Mar. 2, 2001

(51) Int. Cl. C12M 1/34

(52) U.S. Cl. 435/287.2; 435/288.7

(57) ABSTRACT

A biochip reader, superior in signal to noise ratios and economical in costs, comprising a light source, a lens for collimating light emitted by the light source, an optical detector for detecting fluorescent light produced by the excitation light at samples, and an objective lens for condensing fluorescent light produced at a biochip where samples are deposited, wherein the excitation light is projected onto the substrate of the biochip at a critical angle or greater.

### BACKGROUND OF THE INVENTION

1. Field of Invention

This invention relates to a biochip reader, and more particularly, to an improved biochip reader which is superior in signal-to-noise ratio, and is economical in cost.

2. Description of the Prior Art

A biochip, such as DNA chip, comprises several thousand to several hundred thousand types of known DNA segments deposited in arrays on a substrate. When a solution containing an unknown DNA segment is deposited onto a DNA chip, DNA segments of the same type hybridize with each other. This characteristic is utilized to examine a known DNA segment wherein hybridization has taken place, using a biochip reader, to determine the sequence of the unknown DNA segment.

FIG. 1 shows an example of hybridization, wherein six DNA segments DN01, DN02, DN03, DN04, DN05 and DN06 are deposited in arrays on a substrate SB01, thus providing a DNA chip. An unknown DNA segment UN01 is previously marked with a fluorescent marker LM01. When hybridized to a DNA chip, the unknown DNA segment will combine with another DNA segment whose sequence is complementary. For example, unknown DNA segment DN01 will combine with known DAN segment DN01, such as shown by double loops CB01.

Using a biochip reader excitation light is irradiated at the DNA chip thus hybridized, in order to detect fluorescent light produced at the fluorescent marker. Consequently, it is possible to determine which of the known DNA segments the unknown DNA segment has combined with. For example, in an image resulting from scanning a DNA chip SI01, fluorescent light is observed only at the spot where the DNA combination CB01 is produced. Accordingly, fluorescent light is detected only from the spot LD01.

However, dust may collect on the DNA chip when mixing occurs of foreign matter with a liquid in which the unknown DNA segment is hybridized or when subsequent processes are carried out. If the dust is organic, the excitation light will cause the dust to emit fluorescent light which is more intense that the fluorescent light emitted by a site. Accordingly, fluorescent light will act as noise, and the signal to noise ratio (S/N ratio) of the biochip reader is deteriorated.

FIG. 2 shows a conventional biochip reader which minimizes the foregoing problem, wherein light emitted by a light source 1, such as a laser, is condensed by a lens 2 and is reflected by a dichroic mirror 3, so that the light is passed through a pinhole formed in a pinhole plate 4. The excitation light passed through the pinhole is then condensed through an objective lens 5 onto a DNA chip 6 which is a biochip where a plurality of sites are deposited in arrays.

Sites CL01, CL02 and CL03, wherein a plurality of known DNA segments of the same type are placed, are deposited in arrays on DNA chip 6. The excitation light is irradiated at, for example, site CL02. Fluorescent light produced by the excitation light in site CL02 is again passed through the pinhole formed in pinhole plate 4 by way of objective lens 5 and is transmitted through dichroic mirror 3. The fluorescent light passed through dichroic mirror 3 travels through a filter 7 and is condensed onto an optical detector 9, such as a photomultiplier tube, by a lens 8. The DNA chip 6 is scanned by a drive means (not shown). For example, DNA chip 6 is scanned in the direction MV01 so that the excitation light is irradiated at the remaining sites CL01 and CL03 on DNA chip 6.

The biochip reader of FIG. 2 is operated as follows. Excitation light is passed through the pinhole formed in pinhole plate 4. Fluorescent light produced by excitation light in a site is also passed through the same pinhole. The optical system depicted is a confocal optical system, thus providing an improved optical axis resolution. Hence, it is possible to isolate and detect only the fluorescent light that is emitted from the site even when the site is contaminated with dust. Accordingly, it is possible to reduce effect of dust adhering to a site and thereby improve S/N ratio by using a confocal optical system.

In another conventional biochip reader, electrodes are formed on a biochip and a voltage is applied between the electrodes to accelerate hybridization to a DNA chip containing an unknown DNA segment, such as described in Japan PCT Heisei 09/504,910.

In the FIG. 2 biochip reader, a problem exists in that the reader is more expensive and the optical system is more complex since a confocal optical system is used. In the other mentioned reader wherein electrodes are provided, another problem exists in that the reflected light formed at the electrodes act as background light or noise, and hence, the resulting S/N ratio is deteriorated.

### SUMMARY OF THE INVENTION

Accordingly, an object of the invention is to overcome the aforementioned and other disadvantages, problems, and deficiencies of the prior art.

Another object is to provide a biochip reader which is superior in signal-to-noise ratio, and is economical in cost and operation.

The foregoing and other objects are attained by the invention, wherein an excitation light is applied to a sample of, for example, DNA, RNA, protein and/or sugar chain, deposited on a substrate, at a controlled angle which is at the critical angle or greater, and the fluorescent light produced at the sample deposited on the biochip, is detected. Advantageously, by carefully controlling the angle of irradiation by the excitation light, there is substantially total reflection which is detected, and hence signal to noise ratio is improved with economy of construction and operation of the reader.

### BRIEF DESCRIPTION OF THE DRAWING

FIG. 1 is a schematic view depicting an example of hybridization in biochips, which process occurs in conventional and invention biochip readers.

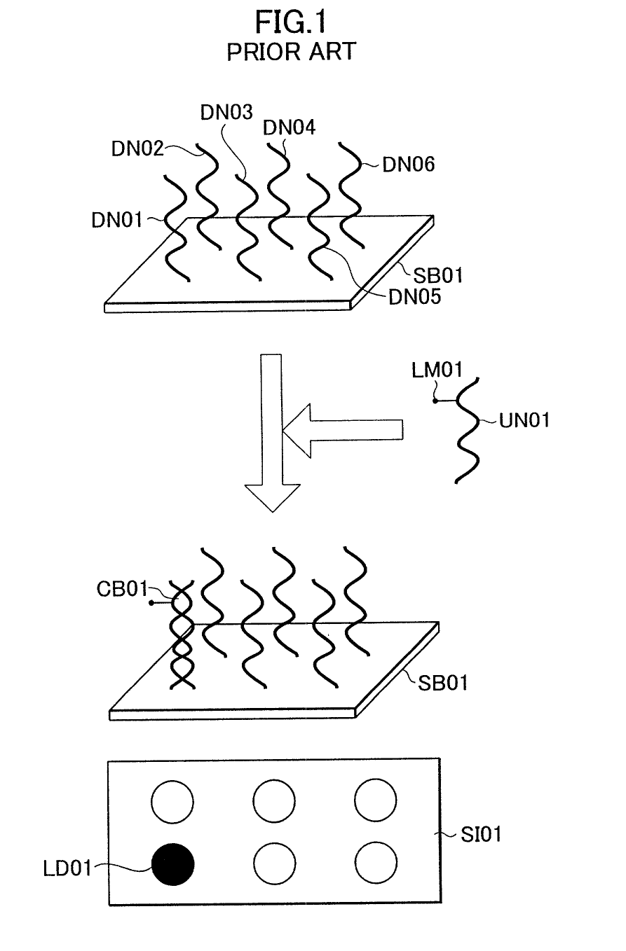

FIG. 2 is a block diagram depicting a conventional biochip reader.

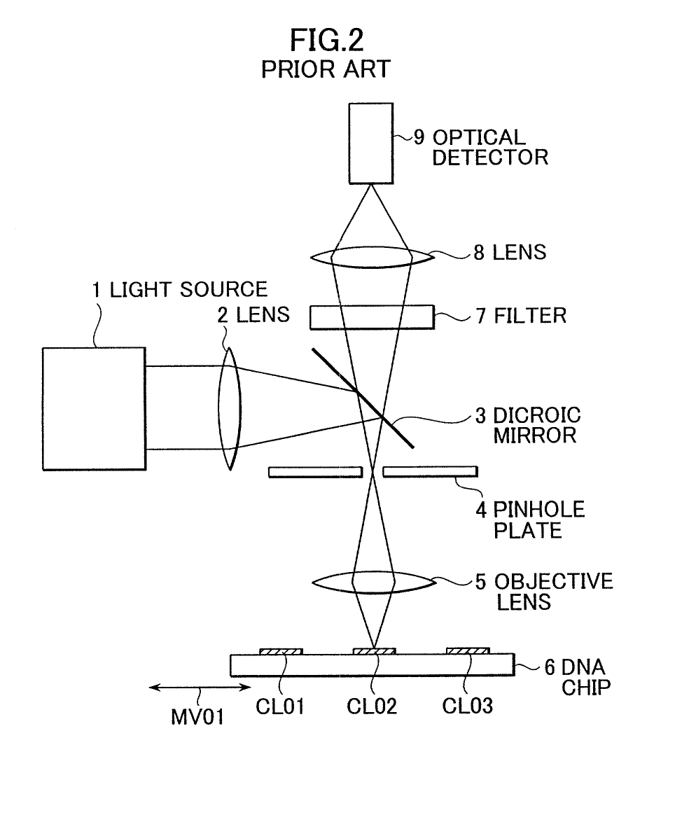

FIG. 3 is a block diagram depicting a first illustrative embodiment of the invention.

FIG, 4 is a partially enlarged view of a site.

FIG. 5 is a block diagram depicting a second illustrative embodiment of the invention.

FIG. 6 is a block diagram depicting a third illustrative embodiment of the invention.

FIG. 7 is another partially enlarged view of a site.

FIG. 8 is a partially enlarged view of a site on a biochip reader of the invention.

### DETAILED DESCRIPTION OF THE PREFERRED EMBODIMENTS

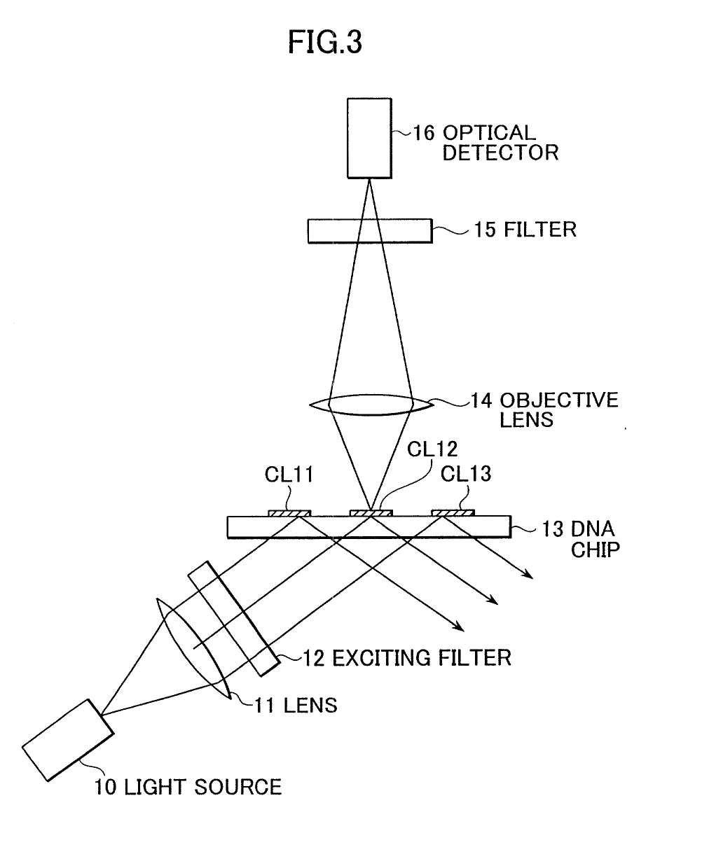

FIG. 3 shows an illustrative embodiment of a biochip reader of the invention, wherein light emitted by a light source 10, such as a laser light source or a white light source, is collimated by a lens 11 and a specified wavelength band of emitted light is then transmitted through an exciting filter 12 as an excitation light. A light source can include the collimating lens and/or filter or both, as desired. The excitation light is then controlledly and obliquely projected onto a DNA chip 13, which is a biochip where a plurality of sites are deposited, from the side of the DNA chip 13 opposite to the side where the sites are deposited, as shown in FIG. 3. Excitation light is irradiated obliquely so that the angle of incidence on DNA chip 13 exceeds or equals the critical angle of the excitation light. Sites CL11, CL12, CL13, in each of which a plurality of DNA segments of the same type are placed as samples, are deposited in arrays on DNA chip 13. Fluorescent light produced in a site is condensed by a filter 15 onto an optical detector 16, such as a CCD, by an objective lens 14.

Operation of the embodiment of FIG. 3 is as follows. Generally light projected through the substrate of DNA chip 13, which is a high refractive index medium, into the air, which is a low refractive index medium, at the critical angle, or greater, results in total reflection at the interface between the two media.

Part of the energy of the incident light travels through the low refractive index medium, however, to become reflection light. Such light that temporarily leaks toward the low refractive index medium side is called “evanescent light’ and a place where the evanescent light occurs is called an evanescent field.

FIG. 4 is a partially enlarged view of a site CL12 of FIG. 3, wherein angle θ is defined as an angle greater than the critical angle, wherein the thickness of the evanescent field “t” is normally equivalent to the wavelength of incident light. Hence, the thickness “t” of an evanescent field is approximately 500 nm when the wavelength of excitation light is set to 500 nm by exciting filter 12. In other words, incident light is temporarily transmitted through the interface into the evanescent field. Accordingly, the incident light is irradiated at sites deposited on the substrate of DNA chip 13. The 500 nm thickness of the evanescent field (discussed above) is significantly greater than a DNA chain but significantly smaller than the thickness (which is several ten microns) of a dust particle DS11 and DS12. Hence, it is possible to reduce the effect of dust and thereby improve the S/N ratio of the reader.

The excitation light is irradiated obliquely at DNA chip 13 from the side thereof opposite the side where sites are deposited in arrays so that the angle of incidence on DNA chip 13 equals or exceeds the critical angle of the excitation light. Accordingly, the evanescent light is irradiated at the sites. Hence, the S/N ratio of the reader is greatly improved over the prior art. In addition, the cost of the biochip reader is greatly reduced since a simple optical system is used in the invention.

The component TE11 is a transparent electrode, which is made of indium-tin oxide (ITO) film, for example. Use of such transparent electrode prevents catoptic light from becoming background noise, hence, the S/N ratio of the invention is improved.

Although the DNA chip is used in FIG. 3, the invention is not limited to DNA chips; in fact, other biochips may be used, such as, for example RNA chips, protein chips, sugar chain chips, etc. In the case of RNA samples, hybridization process is used, in the same manner as with DNA chips. On the other hand, when the samples are of protein or sugar chains, the samples are submitted to antigen-anti-body reaction. In either case, a known sample whose sequence is complementary is combined with an unknown sample marked with a fluorescent marker.

Although a laser light source is used in the example, the excitation light source 16 can be other light sources, such as a white light source, a halogen lamp, a mercury lamp, a xenon lamp, or any other while light source. In the embodiment of FIG. 3, the optical detector 16 is located on the side of the DNA chip 13 where sites are deposited. Alternatively, the optical detector 16 can be located on the other side. Similarly, the excitation light may be disposed on the side whereat the sites are disposed.

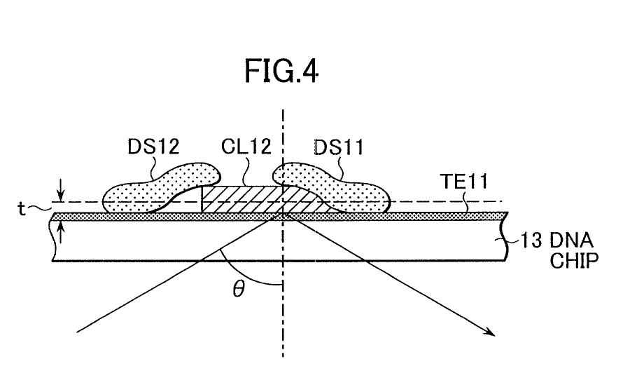

FIG. 5 shows another illustrative embodiment of a biochip reader, wherein light emitted by a light source 10, such as a laser light source, is collimated by a lens 11 and a specified wavelength band of the emitted light is transmitted through an exciting filter 12, as the excitation light. The excitation light is then obliquely irradiated controlledly at sites from the side opposite to the side where the sites are deposited. More specifically, excitation light is irradiated obliquely so that the angle of incidence on DNA chip 13 equals or exceeds the critical angle of the excitation light. Fluorescent light produced by the excitation light in a site is condensed by an objective lens 14a onto an optical detector 16a through a filter 15a. Assume that the substrate of DNA chip 13 is made of, for example glass. Since the refractive index of glass is 1.5, DNA chip 13 produces the same effect as that of immersion, thus improving the numerical aperture (NA) and hence improving the S/N ratio further.

In the embodiment of FIGS. 3 and 5, excitation light is projected onto DNA chip 13 from the side thereof opposite to the side whereat sites are deposited in arrays. Alternatively, the excitation light may be projected from the side whereat sites are deposited.

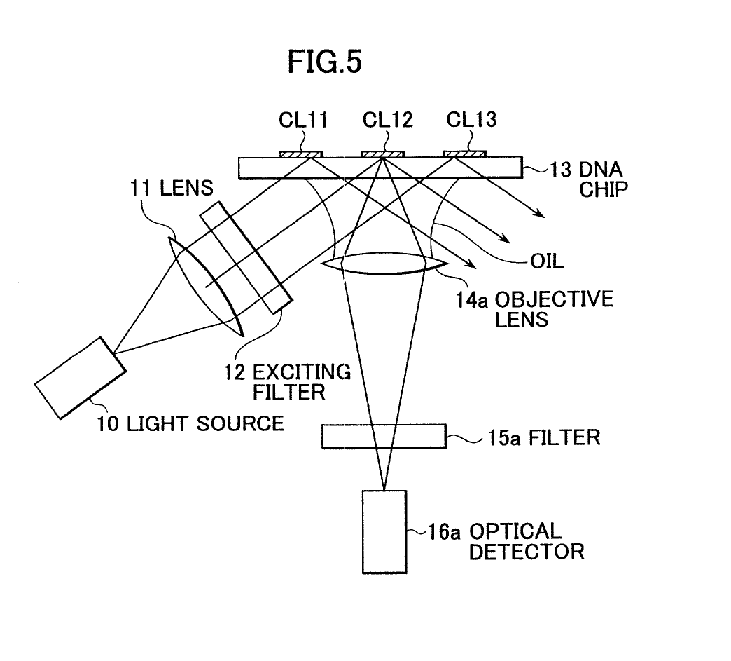

FIG. 6 shows another illustrative embodiment of a biochip reader, wherein light emitted by a light source 10a is collimated by a lens 11a and a specified wavelength band of the emitted light is transmitted through an exciting filter 12a as excitation light. The excitation light is then obliquely projected onto a DNA chip 13 from the side thereof where sites are deposited. Specifically, the excitation light is projected obliquely so that the angle of incidence on the DNA chip 13 equals or exceeds the critical angle of the excitation light. Fluorescent light produced by the excitation light in a site is condensed by an objective lens 14a onto an optical detector 16a through a filter 15a. In this case, the excitation light, projected into the substrate of DNA chip 13, is repeatedly totally reflected within the substrate, as shown by arrow RL11. Hence, fluorescence is caused by evanescent light at a part of DNA chip 13 shown by symbol EL11.

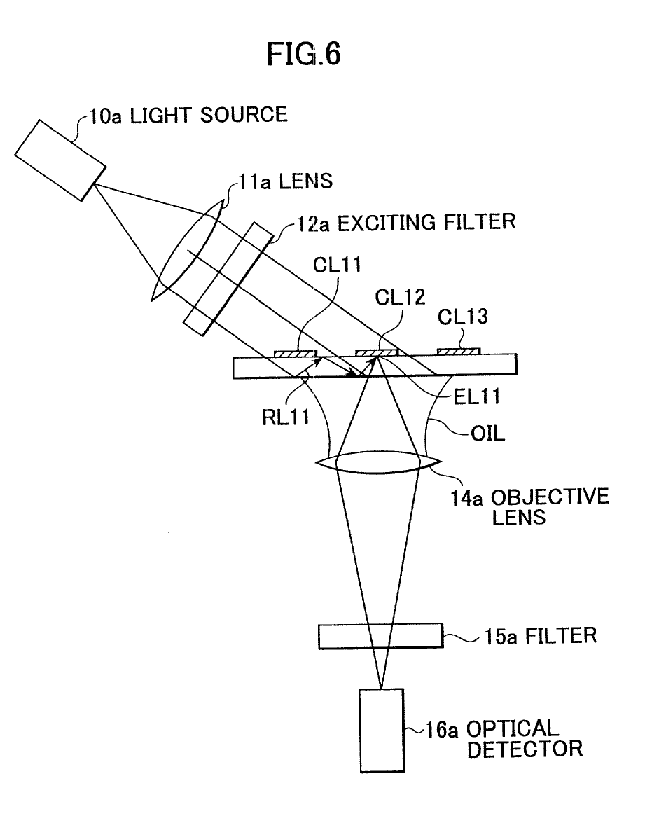

In the embodiments of FIGS. 3 and 5, masks are formed to cover the entire surface of the DNA chip 13, excluding sites. FIG. 7 is a partially enlarged view of a site CL12 of FIG. 3, wherein masks MS11 and MS12 are formed on the substrate of DNA chip 13. By forming the masks, evanescent light can be prevented from being irradiated at dust particles DS11 and DS12 so as to cause the S/N ratio of the reader to be further improved.

In the case where the light; source is a laser light source, speckle noise may occur unless laser light emitted by the light source is adequately condensed. To avoid this problem, laser light focused on the DNA chip 13 may be irradiated onto the surface thereof. Although an area of the DNA chip, as wide as the laser spot, can be tested with a condensed laser beam which is kept still, a wider area of the DNA chip can also be tested by scanning the condensed laser beam. Alternatively, a plurality of laser beams may be used.

In the embodiments of FIGS. 3, 5 and 6, evanescent light is used to irradiate the sites. Alternatively, surface plasmon resonance, which occurs as the result of forming a metal film on the substrate, may be used.

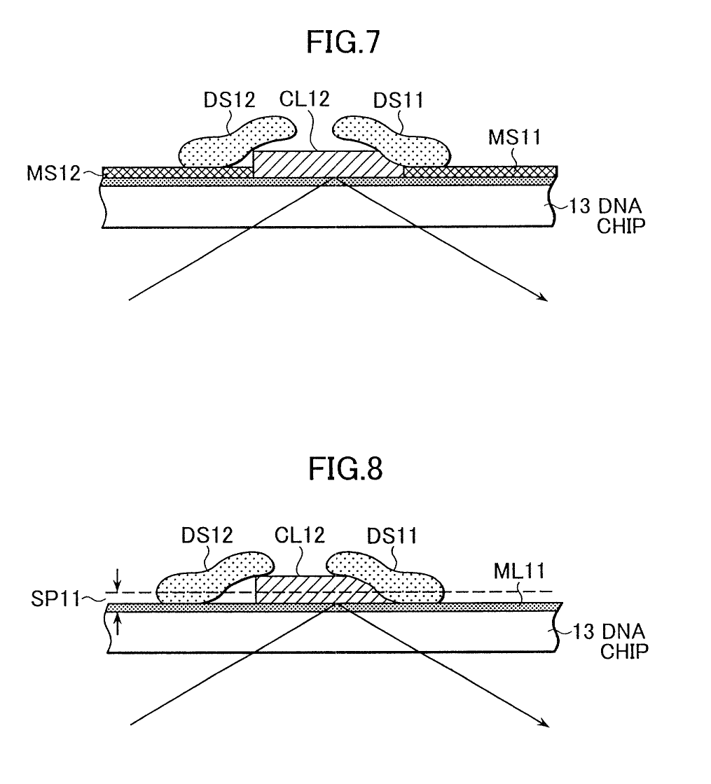

FIG. 8 is a partially enlarged view of a site on a biochip reader of the invention, wherein surface plasmon resonance is the phenomenon that a compressional wave, i.e. electromagnetic wave or or light SP11 occurs within a zone which is as thick as the wavelength of the excitation light on the surface of a thin metal film ML11, when the excitation light is projected through the substrate onto the metal film. Thus, reflected light is effected by the condition of DNA hybridization. The metal film may be used as an electrode for applying voltage to a biochip. In this modification of the embodiment, hybridization can be accelerated by applying a positive voltage to the electrode because the DNA is charged negatively.

The invention has among others the following advantages and effects.

According to one or more aspects of the invention, excitation light is irradiated obliquely and controlledly onto a DNA chip from the side thereof opposite to the side whereat sites are deposited in arrays so that the angle of incidence on the DNA chip equals or exceeds the critical angel of the excitation light, whereby evanescent light is irradiated at the sites. Accordingly, the S/N ratio of the biochip reader is greatly improved and the cost is reduced. Also, the reader is simplified.

Accordingly to another aspect of the invention, an optical detector detects fluorescent light on the side of a biochip opposite to the side whereat samples are deposited. Hence, the numerical aperture NA is improved, and the S/N ratio is further improved.

According to a further aspect of the invention, transparent electrodes are formed on a biochip to prevent catoptric light produced by the formed electrodes from becoming background noise so that the S/N ratio is further improved.

According to a yet further aspect of the invention, laser light emitted by a laser light source is condensed onto and scanned across samples, thereby preventing speckle noise from being produce.

According to another aspect of the invention, masks are formed in areas of the biochip not containing the samples. Hence, evanescent light can be prevented from being irradiated at dust particles. Accordingly, the S/N ratio is further improved.

According to a further aspect of the invention, a metal film is formed on a biochip and surface plasmon resonance is induced by excitation light so that fluorescence occurs on the metal film and a surface plasmon is irradiated at samples, whereby S/N ratio is improved.

According to another aspect of the invention, a metal film is used as an electrode for applying voltage to the biochip so that hybridization is accelerated, with a positive voltage applied to the electrode since the DNA is charged negative.

According to other aspects of the invention, samples under test are DNA or RNa, wherein a known sample whose sequence is complementary is combined with an unknown sample marked with a fluorescent marker as a result of the hybridization. Hence, the sequence of the unknown sample is readily determined.

According to further aspects of the invention, samples under test are protein or sugar chain, wherein a known sample whose sequence is complementary is combined with an unknown sample as a result of antigen-antibody reaction. Hence, the sequence of the unknown sample is readily determined.

The foregoing description is illustrative of the invention. Numerous extensions and modifications thereof would be apparent to the worker skilled in the art. All such extensions and modifications are to be considered to be within the spirit and scope of the invention.

What is claimed is:

1. A biochip reader comprising:
a light source for emitting an excitation light;
a lens for collimating said excitation light;
optical detector means for detecting fluorescent light produced by said excitation light at samples; and
objective lens means for condensing said fluorescent light produced in a biochip where said samples are deposited, onto said optical detector means; wherein
said excitation light is projected onto a substrate of said biochip at a critical angle or greater.

2. The device of claim 1, wherein said optical detector means comprises means for detecting fluorescent light on a side of said biochip where said samples are deposited.

3. The device of claim 1, wherein said optical detector means comprises means for detecting fluorescent light on a side of said biochip opposite to a side where said samples are deposited.

4. The device of claim 1, further comprising means for projecting said excitation light onto said biochip from a side thereof opposite to a side where said samples are deposited.

5. The device of claim 1, further comprising means for projecting said excitation light onto said biochip from a side thereof where said samples are deposited.

6. The device of claim 1, wherein said light source is a laser light source or a white light source.

7. The device of claim 6, wherein said laser light source emits a laser light which is condensed onto said samples and is either kept still or is scanned.

8. The device of claim 1, further comprising a mask formed in areas of said biochip other than areas where said samples are deposited.

9. The device of claim 1, wherein said samples are DNA.

10. The device of claim 1, wherein said samples are RNA.

11. The device of claim 1, wherein said samples are protein.

12. The device of claim 1, wherein said samples are sugar chain.

13. A biochip reader comprising:
a light source for emitting an excitation light;
a lens for collimating said excitation light;
optical detector means for detecting fluorescent light produced by said excitation light at samples; and
objective lens means for condensing onto said optical detector means said fluorescent light produced in a biochip where said samples are deposited; wherein
transparent electrodes are formed on said biochip.

14. The device of claim 13, wherein said transparent electrodes comprise indium tin oxide.

15. The device of claim 13, wherein said light source is a laser light source or a white light source.

16. The device of claim 15, wherein said laser light source emits laser light which is condensed onto said samples and is kept still or is scanned.

17. The device of claim 13, further comprising a mask formed in areas of said biochip other than areas where said samples are deposited.

18. A biochip reader comprising:
a light source for emitting excitation light;
a lens for collimating said excitation light; and
optical detector means for detecting reflection light from a biochip, wherein
a metal film is formed on said biochip, and a surface plasmon is produced by said excitation light on said metal film.

19. The device of claim 18, wherein said metal film is used as an electrode for applying voltage to said biochip.
